(19) United States
(12) Patent Application Publication
(10) Pub. No.: US 2011/0214675 A1
(43) Pub. Date: Sep. 8, 2011

(54) Medical tube holder

(76) Inventor: Louis G. Kost, Owensboro, KY (US)

(21) Appl. No.: 12/660,693

(22) Filed: Mar. 2, 2010

(51) Int. Cl. A61M 16/04

(52) U.S. Cl. 128/207.17

(57) ABSTRACT

A medical tube holder that includes a variable in size head band and a medical tube receiving loop depending therefrom. The loop is formed by a strap that has opposite end portions thereof overlying one another and the terminal ends thereof secured to the headband. The loop is spaced a selected distance from the headband by a necked down provided by the overlapping end portions. A member is slidably mounted on the necked down portion and provides means to selectively vary the size of the loop. The slide member has a locking mechanism movable from one to the other of a slider locked and unlocked state. The holder can be used in association with a bite block and perform the dual function of retaining the medical tube in position on a patient and retain the bite block in position on the medical tube. Also disclosed is a bite block that has an adjustably variable in size hole for receiving and clampingly engaging the medical tube.

### RELATED APPLICATIONS

This application and claims priority from U.S. application Ser. No. 12/384,247 filed on Apr. 2, 2009 hereby incorporated by reference in its entirety.

### FIELD OF INVENTION

This invention relates generally to medical tube holders and more particularly an endotracheal tube holder having a head strap with a loop depending therefrom each of which is variable in size and adjustment thereof independent of one another with a member that is slidably disposed on the loop serves to space the loop a selected distance from the head strap and also vary the size of the loop. The loop being adjustably variable in size can receive tubes of widely varying diameters.

The invention also is directed to the forgoing holder in combination with a bite block wherein the holder performs the dual function of retaining the bite block on the medical tube and holding the medical tube in position on the intubuated patient.

### BACKGROUND OF THE INVENTION

An endotracheal tube (ET) is used to assist in ventilation of a patient during medical procedures. It is inserted into and through a patient's trachea in order to ensure that the airway is maintained in an open condition for cycling of air to and from the lungs. In order to retain the tube in an intended position and orientation for optimal patient benefit, medical personnel have utilized tape but being ad hoc in nature the procedure is subject to variations inherent to different medical personnel training and experience.

ET holders are preferred to tape due to their holding the tube more consistently than tape in the intended position and orientation on the patient As for known, or proposed, tube holders, reference may be had to the following patents and printed publications:
U.S. Patent Application Publication No. 2009/0255538 by Thomson et published Oct. 15, 2009;
U.S. Patent Application Publication No. 2007/0272251 by Hodge published Nov. 29, 2007;
U.S. Pat. No. 7,063,088 by Christopher issued on Jun. 20, 2006; U.S. Pat. No. 5,042,477 by Lewis issued on Aug. 27, 1991; and U.S. Pat. No. 4,437,463 by Ackerman issued on Mar. 20, 1984.

The devices disclosed in the foregoing U.S. Pat. Nos. 5,042,477 and 4,437,463 have a relatively massive assembly that tends to cover the patients mouth (or at least a major portion thereof making it uncomfortable for the patient and difficult for the care giver. Ackerman discloses a device in which the loop for the tube is varied in size by the neck strap.

### SUMMARY OF INVENTION

This invention relates generally to medical tube holders and more particularly an endotracheal tube holder having a head strap with a loop depending therefrom each of which is variable in size and adjustment thereof independent of one another. A member that is slidably disposed on the loop serves to space the loop a selected distance from the head strap and also vary the size of the loop.

The invention also is directed to the forgoing holder in combination with a bite block wherein the holder performs the dual function of retaining the bite block on the medical tube and holding the medical tube in position on the intubuated patient.

The loop being adjustably variable in size can receive tubes of widely varying diameters.

The present invention comprises, consists essentially of, and/or consists of a medical tube holder that includes a variable in size head band and a medical tube receiving loop depending therefrom. The loop is formed by a strap that has opposite end portions thereof overlying one another and the terminal ends thereof secured to the headband. The loop is spaced a selected distance from the headband by a necked down provided by the overlapping end portions. A member is slidably mounted on the necked down portion and provides means to selectively vary the size of the loop. The slide member has a locking mechanism movable from one to the other of a slider locked and unlocked state. The holder can be used in association with a bite block and perform the dual function of retaining the medical tube in position on a patient and retain the bite block in position on the medical tube. Also disclosed is a bite block that has an adjustably variable in size hole for receiving and clampingly engaging the medical tube.

A principal object of the present invention is to provide an ET holder in which the head band and loop for the endotracheal tube are adjustably variable in size independent of one another

Another object of the present invention is to provide an ET holder that is simple in construction, has relatively few parts and a relatively small amount of mass in front of the mouth of an intubuated patient.

A further principal object of the present invention is to provide an ET holder in which a loop for the tube is adjustably variable in size and spaced a selected distance from the lip engaging portion of the head band

In keeping with the forgoing there is provided in accordance with the present invention an ET holder comprising a head band, a strap having opposite end portions overlying one another with the terminal ends thereof secured to said head band providing a loop that is spaced a selected distance from the headband by a necked down portion of the loop, a member slidably mounted on said necked down portion providing means to selectively vary the size of the loop and releasably lock means on said slide member cooperatively engagable with said necked down portion of the loop to selectively lock and unlock the slider to respectively prevent and allow movement of the slider relative to the loop.

The tube holding loop is spaced a selected distance from the head band portion to which it is secured whereby only the loop portion of the holder is in front of the patients mouth and also the head band portion, to which the loop is attached, is positioned to abut only an upper portion of the patients lip proximate their nose.

Each of the head band and tube holding loop are selectively adjustable in size with adjustment of one being independent of adjustment of the other.

Other objects, features, and advantages of the invention will be apparent with the following detailed description taken in conjunction with the accompanying drawings showing a preferred embodiment of the invention.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1 side oblique view of an endotracheal tube holder provided in accordance with an aspect of the present invention;

FIG. 2 is a frontal oblique view of the holder shown in FIG. 1;

FIG. 3 is a side oblique view of the holder shown in FIGS. 1 and 2 grippingly engaging an endotracheal tube and a portion of a bite block retaining the same in a preselected location thereon;

FIG. 4 is a side view of the bite block shown in FIG. 3;

FIG. 5 is a front view of FIG. 4;

FIG. 6 is a view illustrating a specific bite block provided by applicant that on and grippingly engages the airway tube without the presence of a tube holder;

FIG. 7 is an exploded, part sectional, view of the bite block shown in FIG. 6;

FIG. 8 is a is a sectional view taken essentially along line 3-3 of FIG. 7; and

FIG. 9, similar to a portion of FIG. 7, illustrates modifications to a portion of the bite block shown in FIG. 7.

### DETAILED DESCRIPTION OF THE PREFERRED EMBODIMENT

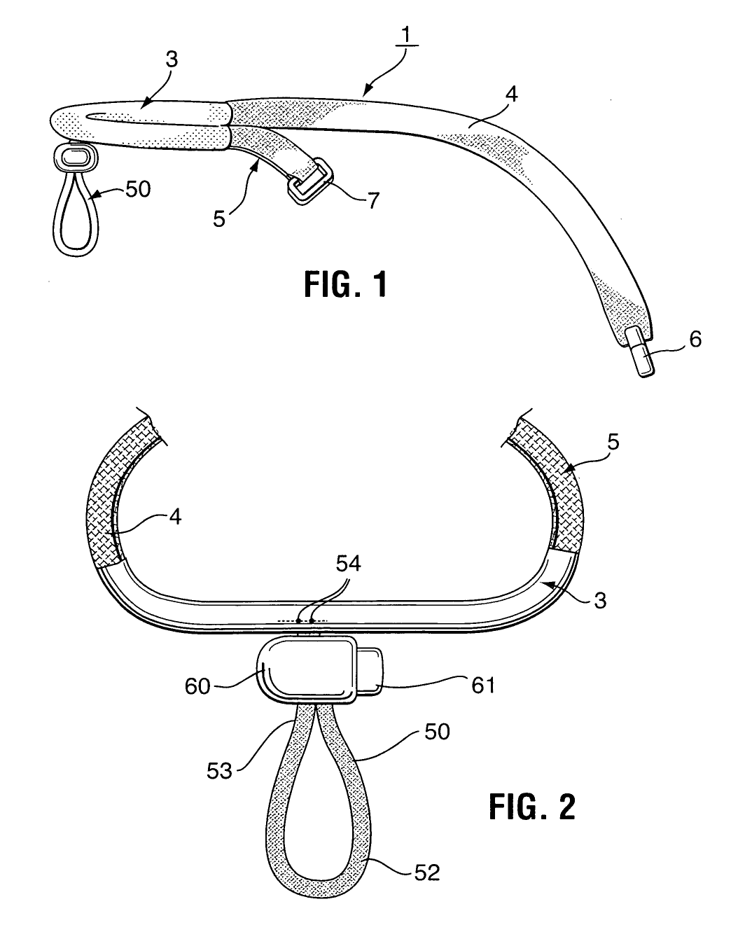

Illustrated in FIGS. 1 and 2 is an endotracheal tube holder comprising a head strap 1 having an adjustably variable in size loop 50 depending therefrom. FIG. 1 illustrates the head strap 1 lying in a first plane and the loop 50 in a second plane with such planes being at an angle to one another. The preferred angle approximates 90 degrees such that with the head strap going around the patients head, just above the ears, the loop overlies the patients mouth having an at rest position that places little or no twisting force on an ET passing there through. The head strap forms an adjustably variable in size head band.

The head strap includes a central portion 3, from which the loop depends, connected at opposite ends thereof to respective strap portions 4, 5 the free outer ends of which have respectively an anchoring tab 6 and an open eye 7. The free end of the strap portion 4 can be threaded through the open eye 7 and the tab 6 then anchored to that strap portion to form a closed band. The tab and strap for example may be a hook and loop fastener or the tab could if desired be a hock that snags the strap portion at preselected or at an infinite number of positions there along. At least one of respective portions 4, 5 is elastically stretchable thereby permitting the strap to be suitably tensioned for a comfortable fit and remain in place on the patient.

The strap portion 3 is for example circular in outline cross-section shape and preferably has, at least, an outer layer of a soft cushioning material for patient comfort.

The loop 50 is formed by a rectangular in cross-section strap 52 having opposite end portions overlying one another providing a necked down portion 53 intermediate the loop and the head strap to which it is secured. The strap 52 terminates in opposite ends that are securely anchored as at 54 to the head band portion 3. The strap 52 is made of a non-toxic, slip and moisture resistant material.

A member 60 is mounted on the loop necked down portion 53 spacing the tube receiving portion thereof a selected distance from the head strap portion 3. The preferred spacing locates the head band portion 3 on the patients upper lip at the patients nose or proximate thereto.

The member 60 is selectively slidable along the loop necked down portion 52 and thereby permits one to selectively vary the size of the loop. In the loops largest size an endotracheal tube is readily insertable but once the tube is suitably located therein the size is reduced bringing the inner face of the strap 52 into pressured engagement with the outer peripheral surface of the tube. The flat face of the strap provides large surface contact resisting sliding of the loop along the tube.

Means for securing in place comprises a spring loaded finger engagable lock and release mechanism wherein the release button 61 is mounted on the member 60 and has a portion cooperatively engaging the strap 52 to releasably engage the same to selectively restrain and allow sliding movement of the member 60.

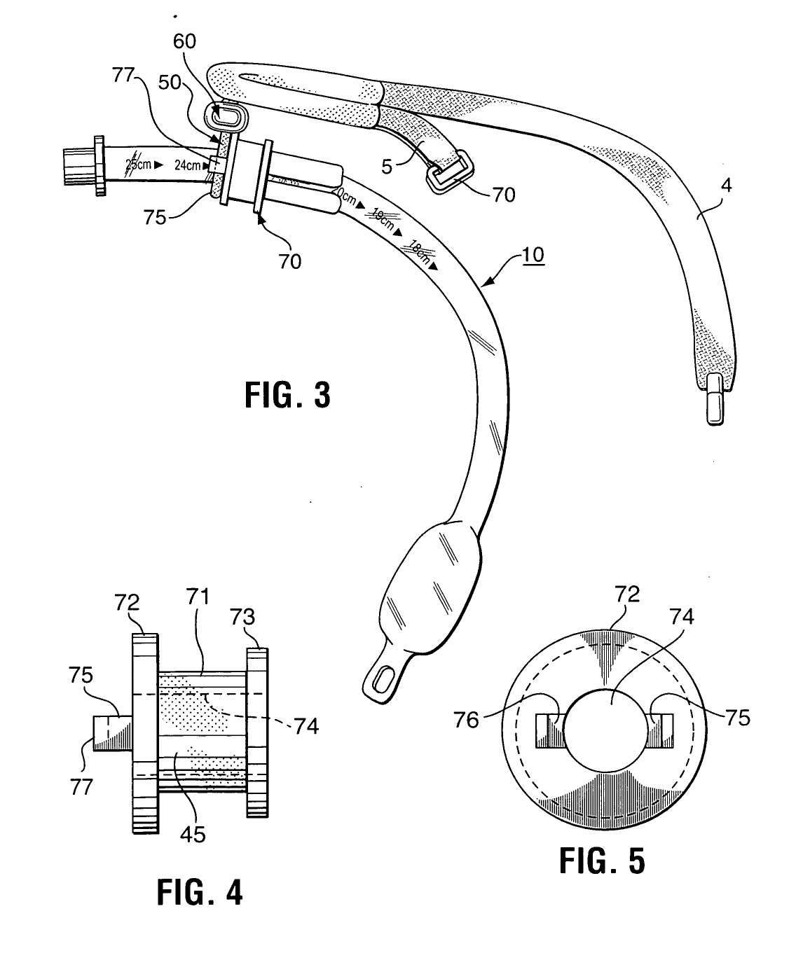

Illustrated in FIG. 3 is a known airway tube 10 with a bite block 70 thereon. The tube holder, as previously described, retains the tube in position on the patient by a medical attendant tightening the loop around the tube of a suitably intubuated patient but in this embodiment the loop 50 of the tube holder also restrains the bite block from movement along the tube.

The bite block 70 has a hub portion 71 with flanges 72, 73 at respective opposite ends thereof. There is a hole 74 through which a medical tube can be inserted. The flange 72 extends radially further from the hub than flange 73 and during use is located outside of the patients mouth. A pair of lugs 75, 76 project from the flange 72 and are located respectively on opposite sides of the hole 74 at positions adjacent thereto. These lugs if desired may have an end tab 77 directed outwardly and spaced from the flange so that the loop strap 52 will fit there between. In this embodiment the bite block lugs are held captive between the inner surface of the loop strap 52 and the outer surface of the medical tube 10 as the loop is brought into tight pressured contact with the outer peripheral surface of the tube. In this manner the holder loop 50 anchors the bite block to the tube and at the same time holds the tube in position on the patient. The through hole 74 is a one size fits all with flexing of the lugs 75, 76 allowing the bite block to accommodate differing tube diameters.

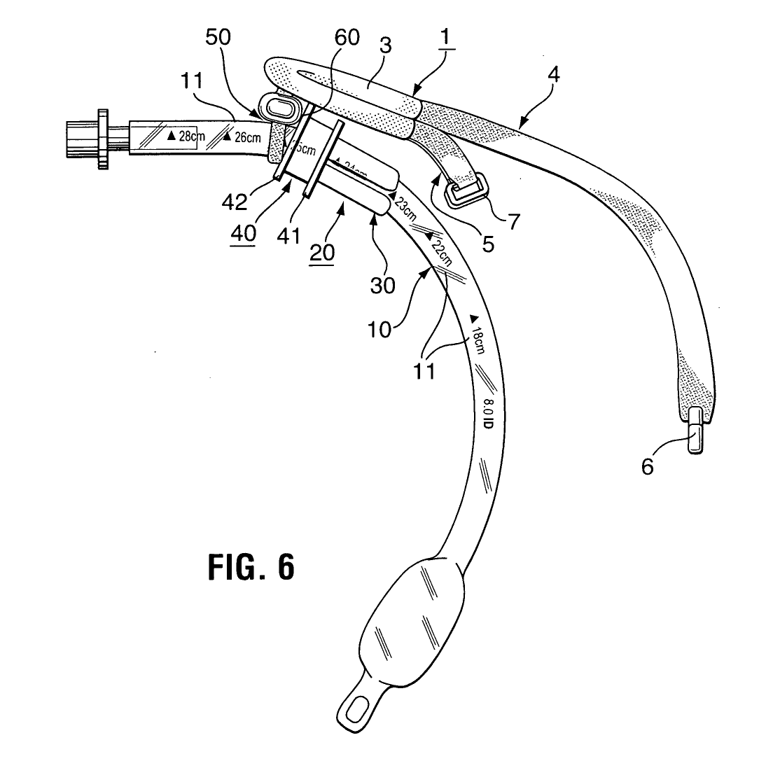

FIG. 6 is similar to FIG. 3 but illustrates applicants specifically constructed bite block 20 on a known airway tube 10. The bite block has an adjustably variable in size through hole that allows the tube to pass there-through. Airway tubes are available with differing outer diameters dictated generally by whether the patient is an adult or child, male or female. At least a portion of the through hole is selectively adjustably variable in diameter from a maximum diameter, that is slightly greater than the outer diameter of a tube to used in association therewith, to a minimum diameter that is smaller than the outer diameter of such tube. The portion that can be reduced in diameter is resiliently biased to it's maximum diameter which is its normal at rest state.

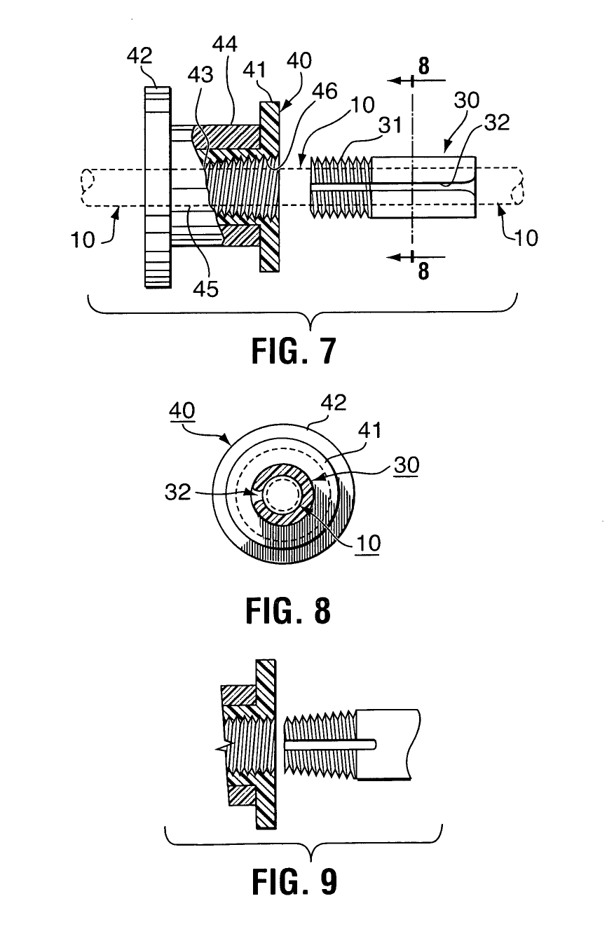

Referring now in more detail to FIGS. 6 to 9 the bite block 20 comprises respective first and second components 30, 40 in which the first component 30 is an elongate open ended tubular sleeve having an externally threaded portion 31 extending from one end of the sleeve in a direction toward the other end. There is a slit 32 in the wall of the sleeve that extends from the above mentioned one end in a direction toward the other end. In the preferred embodiment, illustrated in FIG. 7, the slit 32 extends along the entire length of the tubular sleeve while in the embodiment illustrated in FIG. 9 the slit extends only along a portion of the length of the sleeve. The purpose of the slit is to accommodate changing the internal diameter of the sleeve from it's normal at rest state of maximum diameter that is greater than the outer diameter of the tube 10 being used in association therewith to a minimum diameter that is smaller than that outer diameter of the tube 10. At this minimum diameter the bite block tightly grips the tube 10.

The sleeve 30 is preferably made of a transparent plastics material (preferably food grade or medical grade) permitting progressive tube length designations 11 imprinted on the tube 10 to be seen through the wall of the sleeve. The wall thickness of the sleeve is chosen taking into account the stiffness of the material such that the sleeve internal diameter can be readily varied using only finger grip to rotate the components relative to one another about the longitudinal axis of the sleeve 30. The characteristics of the material such as resilience, flexibility and elasticity are considered and chosen so that the sleeve is resiliently biased to a normal at rest maximum diameter and permitting repeated usage by staying within the elastic limits as the sleeve is deformed.

The second component 40 is a rigid or relatively stiff body member made of a plastics, metal, or rubber like material or combinations thereof. The body has spaced apart ridges 41, 42 interconnected by a hub 43. Ridge 42 projects further from the hub than the ridge 41 and when in use abuts against the outer upper and lower lip portions of the patient. The hub 43 has a layer 44 of soft resilient material located, at least, in appropriate positions for abutting the patients respective upper and lower teeth. This padding of softer material may, for example, be a suitable plastics material such as a thermoplastic, elastomer material, plastisol, neoprene or a rubber or rubber like material that protects the teeth from chipping or other damage. The ridges 41, 42 may be circular flanges with one having a larger diameter than the other as shown and together with the hub interconnecting the same provide a body having the general appearance of a spool. The hub of the spool is surrounded by an annular cushioning member. Alternatively the flanges 41 and 42, at minimum, could each consist of a pair of ribs projecting radially in opposite directions away from the hub.

There is an open ended hole 45 through the body member 40 with at least a portion thereof having internal screw threads 46. In the embodiment illustrated in FIG. 2 these threads are located in a tapered portion decreasing in diameter in a direction from flange 41 toward flange 42. The sleeve 30 external threads 31 mate with the threads 46 and as components 30, 40 are rotated relative to one another to increase penetration of component 30 into component 40 the tapering of the threads causes the internal diameter of the sleeve to progressively decrease in size. The width of the slit 32 is so chosen as to allow a selected reduction in diameter. The amount of taper of the threads is chosen such that the reduction in diameter is sufficient for the sleeve to tightly grasp the tube 10 used in association therewith and without exceeding the elastic limit of the sleeve.

To use the bite block the sleeve is slide over the airway tube just past the patients teeth after such tube has placed in the trachea. While holding the sleeve the body portion of the bite block is slid onto the tube and rotated to thread it onto the sleeve. Rotation of the body portion is stopped when the bite block is secure on the tube and the patients teeth are positioned on the soft outer hub portion of the body member.

By having the bite block tightly grasp the airway tube it is independent and secure without the need for a separate anchoring means that can hinder and obstruct access to the airway.

The bite block is preferably made of a transparent (clear) material and this allows the health care practitioner to visually see the airway features and centimeter markings on the oral or tracheal tube making it easier to note the correct position of the tube.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

1. A medical tube holder comprising a head band, a strap having opposite end portions overlying one another with the terminal ends thereof secured to said headband providing a loop that is spaced a selected distance from the headband by a necked down portion of the loop, a member slidably mounted on said necked down portion providing means to selectively vary the size of the loop and lock means on said slide member cooperatively engaging said necked down portion of the strap and movable from one to the other of a slider locked and unlocked state.

2. The medical tube holder as defined in claim 1 wherein said head band is adjustably variable in size.

3. The medical tube holder as defined in claim 2 wherein said loop forming strap is rectangular in cross-sectional shape.

4. The medical tube holder as defined in claim 1 wherein said head band comprises a strap that includes a central portion from which the tube holding loop depends and first and second strap portions secured to and extending from respective opposite terminal ends of said central portion, said first and second strap portions being interconnectable and thereby form a variable in size head band.

5. The medical tube holder as defined in claim 4 wherein said head band central portion is circular in outline cross-sectional shape and has at least an outer layer of a soft cushioning material.

6. The medical tube holder as defined in claim 4 wherein at least one of said first and second head strap portions is made of an elastically stretchable material.

7. The medical tube holder as defined in claim 1 wherein said headband and loop lie in respective first and second planes disposed at a selected angle to one another.

8. The medical tube holder as defined in claim 7 wherein said selected angle is approximately 90 degrees.

9. A medical tube holder comprising a head band, a strap having opposite end portions overlying one another with the terminal ends thereof secured to said headband providing a medical tube receiving loop that is spaced a selected distance from the headband by a necked down portion of the loop, a member slidably mounted on said necked down portion providing means to selectively vary the size of the loop to receive and be brought into pressured engagement with the medical tube and lock means on said slide member cooperatively engaging said necked down portion of the strap and movable from one to the other of a slider locked and unlocked state and a bite block that slidably mounts on said medical tube, said bite block having a flange that abuts the outer lip portions of the patient during use and including at least one lug projecting from said flange for being captively located between an inner surface portion of the loop and outer surface of the medical tube.

10. A medical tube holder comprising a head band, a strap having opposite end portions overlying one another with the terminal ends thereof secured to said headband providing a loop that is spaced a selected distance from the headband by a necked down portion of the loop, a member slidably mounted on said necked down portion providing means to selectively vary the size of the loop and lock means on said slide member cooperatively engaging said necked down portion of the strap and movable from one to the other of a slider locked and unlocked state and a bite block slidably mountable on said medical tube, said bite block having an adjustably variable in size hole through which the medical tube is inserted therethrough.

11. The medical tube holder and bite block as defined in claim 10 wherein said bite block comprises:
(A) a first relatively rigid body member, an open ended hole of selected length extending through said body member, internal screw threads on at least a portion of the length of said hole; and
(B) a second member comprising an elongate open ended sleeve having a preselected wall thickness and in a normal at rest state a preselected internal diameter, a slit in said wall that extends from one end thereof in a direction toward the opposite end, external screw threads on said sleeve extending from said one end in a direction toward said opposite end, said internal and external threads being inter-engagable in mating relation detachably interconnecting said sleeve and said body member, a selected one of said internal and external threads progressively decreasing in diameter so that with the bite block in an assembled state the sleeve internal diameter gradually decreases to a selected minimum diameter smaller than the outer diameter of a tube associated therewith as the sleeve is threaded into the body member.

12. The medical tube holder and bite block as defined in claim 11 wherein said slit extends at least throughout the length of said external threads on said sleeve.

13. The medical tube holder and bite block as defined in claim 11 wherein said slit extends throughout the entire length of said sleeve.

14. The medical tube holder and bite block as defined in claim 11 wherein at least said sleeve is made of a transparent plastics material.

15. The medical tube holder and bite block as defined in claim 11 wherein the internal threads in said body member progressively decrease in diameter.

16. The medical tube holder and bite block as defined in claim 11 wherein said body member comprises a hub, a first rib means on one end thereof and a second rib means on the opposite end, said fist and second rib means being spaced apart a selected distance from one another and projecting outwardly in a direction radially from said hub with said first rib means projecting a further distance than said second rib means.

17. The medical tube holder and bite block as defined in claim 16 wherein said first rib means comprises a first annular flange.

18. The medical tube holder and bite block as defined in claim 17 wherein said second rib means comprises a second annular flange.

19. The medical tube holder and bite block as defined in claim 18 wherein said first and second annular flanges are circular in outline shape about an axis parallel to the axis of the hole through said body member.

20. The medical tube holder and bite block as defined in claim 16 wherein said hub has a resiliently outer surface.

21. The medical tube holder and bite block as defined in claim 11 wherein said body member comprises a cylindrical hub, a first flange and a second flange disposed respectively at respective opposite ends of said hub, said flanges being spaced apart a selected distance from one another and projecting in a direction radially from the hub with said first flange projecting further than said second flange and a resilient outer surface on said hub.

22. The medical tube holder and bite block as defined in claim 21 wherein said soft outer surface surrounds said hub.
